(19) United States
(12) Patent Application Publication
(10) Pub. No.: US 2022/0362536 A1
(43) Pub. Date: Nov. 17, 2022

(54) In-Line Sampling Apparatus for Bodily Fluids

(71) Applicant: C. R. Bard, Inc., Franklin Lakes, NJ (US)

(72) Inventors: Hoang D. Nguyen, Marietta, GA (US); Serena R. Agrawal, Salt Lake City, UT (US); Kuilin Lai, Watkinsville, GA (US); Michal Tourchak, Atlanta, GA (US); D. Claire Gloeckner, Lilburn, GA (US); Teri Dailey, Mansfield, GA (US); Caroline Ottinger, North Bend, WA (US)

(21) Appl. No.: 17/728,752

(22) Filed: Apr. 25, 2022

Related U.S. Application Data
(63) Continuation of application No. 63/187,246, filed on May 11, 2021.

(51) Int. Cl. A61M 39/02; A61B 10/00

(52) U.S. Cl. CPC A61M 39/02; A61B 10/007; A61M 25/0017

(57) ABSTRACT

A drainage liquid sampling system is disclosed and includes a connector couplable with a drainage tube of a drainage system, the connector defining an inlet port, an outlet port, and a sample port, where the inlet port, the outlet port, and the sample port are in fluid communication with each other, and a sample container couplable with the sample port. A valve within the connector selectively allows and prevents flow of drainage liquid into the sample container. A cap couplable to the sample container also includes a valve. A system for draining liquid from a patient includes the drainage liquid sampling system.

### PRIORITY

This application claims the benefit of priority to U.S. Provisional Application No. 63/187,246, filed May 11, 2021, which is incorporated by reference in its entirety into this application.

### BACKGROUND

The draining of liquid such as urine from a patient may include the use of a liquid drainage system including a flexible drainage tube extending from a drainage catheter to a collection container. Typical catheters include indwelling catheters, Foley catheters, balloon catheters, peritoneal drainage catheters, or the like, and are configured to be inserted into an orifice within the body of a patient to drain a liquid therefrom. In some instances, it may be desirable to obtain a sample of the drainage liquid. As the current drainage systems define a closed pathway from the catheter to the collection container, obtaining a sample of the drainage liquid requires interrupting the drainage process. As such, providing an apparatus and process to obtain a liquid sample without interrupting the drainage process would be advantageous for the clinician.

### SUMMARY

Briefly summarized, disclosed herein is a drainage liquid sampling system. The system includes a connector couplable with a drainage tube of a drainage system. The connector defines an inlet port, an outlet port, and a sample port, where the inlet port, the outlet port, and the sample port are in fluid communication with each other, and a drainage liquid flowing through the drainage tube flows through the connector from the inlet port to the outlet port. The sampling system further includes a sample container couplable with the sample port.

The connector may include a connector valve disposed in line with the sample port. The connector valve may be selectively configurable between an “open” state and a “closed” state, such that drainage liquid is allowed to through the sample port when the connector valve is in the “open” state, and drainage liquid is prevented from flowing through the sample port when the connector valve is in the “closed” state. In some embodiments, the connector valve is configured to automatically open upon coupling of the sample container with the connector, and automatically close upon decoupling of the sample container from the connector.

The sampling system may further include a cap couplable with the sample container, where the cap is configured to sealably couple with an open end of the sample container. The cap may include a cap valve selectively configurable between an “open” state and a “closed” state, such that drainage liquid is allowed to flow into the sample container when the cap valve is in the “open” state, and drainage liquid is prevented from flowing out of the sample container when the cap valve is in the “closed” state. In some embodiments, the cap is configured to couple with the connector. In such embodiments, the cap valve is configured to automatically allow drainage liquid to flow into the sample container upon coupling of the cap with the connector, and automatically prevent drainage liquid from flowing out of the sample container upon decoupling of the cap from the connector.

The connector valve may include a connector septum disposed across the sample port. The connector septum may be deflectable, such that when the connector septum is in a deflected state, the connector valve is in the “open” state, and when the connector septum is in a non-deflected state, the connector valve is in the “closed” state. In some embodiments the connector septum includes a connector slit extending through the connector septum. The connector slit is configurable between an “open” condition and a “closed” condition, such that the connector slit is in the “open” condition when the connector septum is in the deflected state, and the connector slit is in the “closed” condition when the connector septum is in the non-deflected state.

The cap valve may include a cap septum disposed across an opening at a distal end of the cap. The cap septum may be deflectable, such that when the cap septum is in a deflected state, the cap valve is in the “open” state, and when the cap septum is in a non-deflected state, the cap valve is in the “closed” state. In some embodiments, the cap septum includes a cap slit extending through the cap septum. The cap slit is configurable between an “open” condition and a “closed” condition, such that the cap slit is in the “open” condition when the cap septum is in the deflected state, and the cap slit is in the “closed” condition when the cap septum is in the non-deflected state. In some embodiments, the cap includes a protrusion disposed within the opening of the cap, and the protrusion is configured to deflect the cap septum and connector septum upon coupling of the cap with the connector.

One or more portions of the sampling system may include an anti-microbial coating. The connector septum and/or the cap septum may include the anti-microbial coating.

Also disclosed herein is a drainage system for draining liquid from a patient. The drainage system includes a drainage tube configured to couple with a drainage catheter at a distal end of the drainage tube, a collection container coupled to the drainage tube at a proximal end of the drainage tube, and the drainage liquid sampling system as summarized above.

Also disclosed here is a method of draining liquid from a patient. The method includes coupling a distal end of a drainage tube with a catheter, where the catheter is in fluid communication with a drainage liquid source within a patient. The method further includes establishing a passive flow of the drainage liquid from the patient along the drainage tube to a collection container and obtaining a sample of the drainage liquid within a sample container coupled to a connector disposed in line with the drainage tube.

In some embodiments of the method, the connector includes a connector valve, and the method further includes opening the connector valve.

The method may further include coupling the sample container to the connector, and in some embodiments, coupling the sample container to the connector causes the connector valve to open. Coupling the sample container to the connector may include coupling the sample container to a cap and coupling the cap to the connector.

In some embodiments of the method, the cap includes a cap valve, and the method further includes opening the cap valve. In further embodiments, coupling the cap to the connector causes the cap valve to open.

These and other features of the concepts provided herein will become more apparent to those of skill in the art in view of the accompanying drawings and the following description, which describe particular embodiments of such concepts in greater detail.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a drainage system including a drainage liquid sampling system, in accordance with some embodiments disclosed herein.

FIG. 2A is a cross-sectional illustration of the drainage liquid sampling system of FIG. 1 in a disconnected state, in accordance with some embodiments disclosed herein.

FIG. 2B is a cross-sectional illustration of a portion of the drainage liquid sampling system of FIG. 2A in a connected state, in accordance with some embodiments disclosed herein.

FIG. 3A is a cross-sectional illustration of another embodiment of a drainage liquid sampling system in a disconnected state, in accordance with some embodiments disclosed herein.

FIG. 3B is a cross-sectional illustration of the drainage liquid sampling system of FIG. 3A in a connected state, in accordance with some embodiments disclosed herein.

FIG. 4A is a cross-sectional illustration of another embodiment of a drainage liquid sampling system in a disconnected state, in accordance with some embodiments disclosed herein.

FIG. 4B is a cross-sectional illustration of the drainage liquid sampling system of FIG. 4A in a connected state, in accordance with some embodiments disclosed herein.

FIG. 5A is a cross-sectional illustration of another embodiment of a drainage liquid sampling system in a disconnected state, in accordance with some embodiments disclosed herein.

FIG. 5B is a cross-sectional illustration of the drainage liquid sampling system of FIG. 5A in a partially connected state, in accordance with some embodiments disclosed herein.

FIG. 5C is a cross-sectional illustration of the drainage liquid sampling system of FIG. 5A in a fully connected state, in accordance with some embodiments disclosed herein.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to” and “coupled with” refer to any form of interaction between two or more entities, including mechanical, electrical, magnetic, electromagnetic, fluid, and thermal interaction. Two components may be connected or coupled with each other even though they are not in direct contact with each other. For example, two components may be coupled with each other through an intermediate component.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

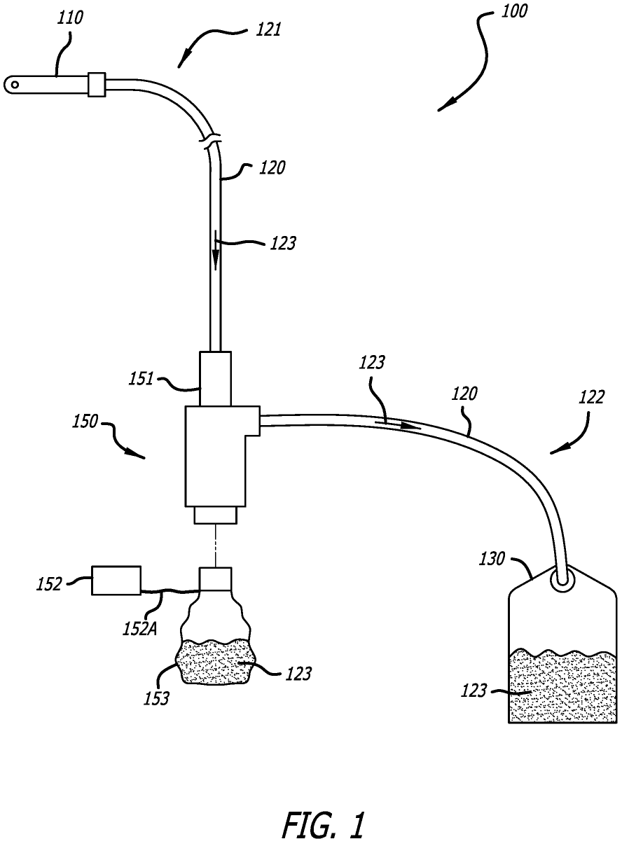

FIG. 1 shows an exemplary drainage system (“system”) 100, including a catheter 110, a drainage tube 120, a collection container 130, and a drainage liquid sampling system 150. The catheter 110 includes an eyelet 112 that provides fluid communication with a lumen of the catheter 110, and is configured to access a source of liquid 123 within a patient. In general, the system 100 provides a drainage pathway to transport liquid 123 from the catheter 110 to the collection container 130.

A distal portion 121 of the drainage tube 120 extends from the catheter 110 to the sampling system 150, and a proximal portion 121 of the drainage tube 120 extends from the sampling system 150 to the collection container 130. The sampling system 150 includes a connector 151, a cap 152, and a sample container 153. In use, the liquid 123 from the patient may flow from the catheter 110, through the distal portion 121 of the drainage tube 120, through the connector 151, and further through the proximal portion 122 of the drainage tube 120 to the collection container 130. In further use, a portion of the drainage liquid 123 may be flow into the sample container 153 as described in detail below. The drainage tube 120 can be formed of rubber, plastic, polymer, silicone, or similar suitable material. The collection container 130 can include a rigid container, a flexible collection bag, or similar suitable container for receiving the liquid 123 (e.g., urine) drained from the catheter 110. In some embodiments, the cap 152 may be attached to the sample container 153 via a tether 152A.

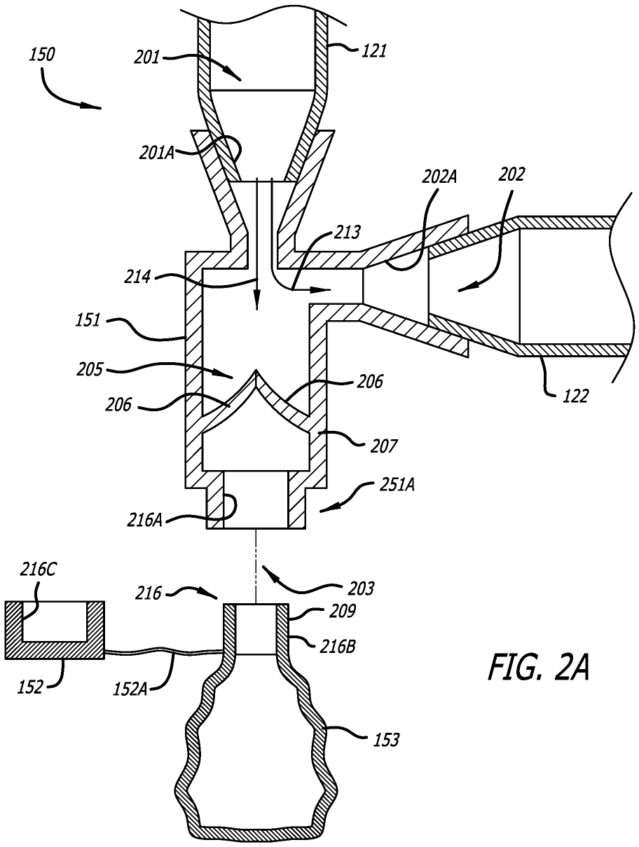

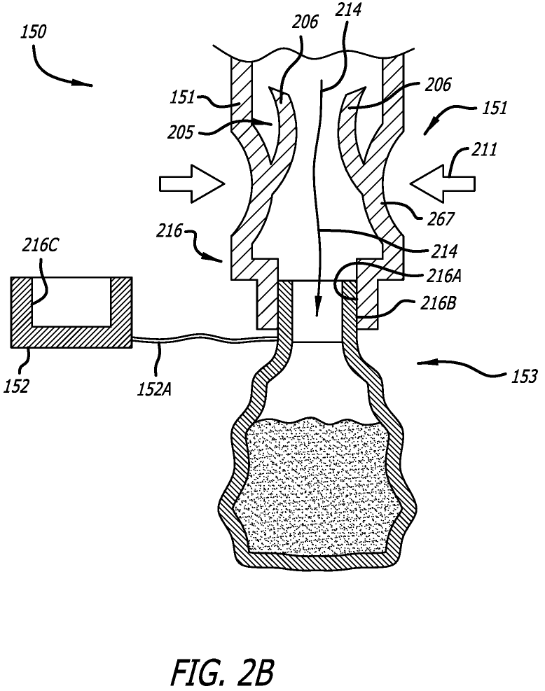

FIGS. 2A-2B illustrate the drainage liquid sampling system 150 including the connector 151, the cap 152, and the sample container 153. FIG. 2A shows the sampling system 150, i.e., the connector 151, the cap 152, and the sample container 153, in a disconnected state. FIG. 2B shows the connector 151 and the sample container 153 in a connected state. The sample container 153 may be flexible container such as a bag or a rigid container such as a bottle. In some embodiments, the sampling system 150 may include more than one sample container 153. In other embodiments, the sample container 153 may be one of a plurality of sample containers, where the plurality includes sample containers of different sizes.

With reference to FIG. 2A, the connector 151 includes an input port 201 in fluid communication with an output port 202 via a main flow path 213. In use, drainage liquid 123 flows along the main flow path 213 as it flows from the catheter 110 toward the collection container 130. As shown in FIG. 1, the inlet port 201 is coupled with (or is couplable with) the distal portion 121 of the drainage tube 120. In other words, the inlet port 201 is configured to couple with the drainage tube 120. In some embodiments, the inlet port 201 may be fixedly attached to the drainage tube 120. In other embodiments, the clinician may attach the drainage tube 120 to the inlet port 201 prior to performing a drainage process. In a similar fashion, the outlet port 202 is configured to couple with the proximal portion 122 of the drainage tube 120.

In some embodiments, the inlet and outlet ports 201, 202 may include tapered or conical portions 201A, 202A, respectively to facilitate attachment of the inlet and outlet ports 201, 202 to an outside surface the drainage tube 120. In other embodiments, the inlet and outlet ports 201, 202 may include other structural elements (e.g., barbs, ribs, sockets, etc.) to facilitate attachment of the inlet and outlet ports 201, 202 to the outside surface or an inside surface of the drainage tube 120.

The connector 151 defines a sample flow path 214 extending from the inlet port 201 to a sample port 203. In other words, the connector 151 defines a bifurcation of the drainage tube 120. In use, the liquid 123 may flow from the inlet port 201, along the sample flow path 214, and through the sample port 203 to the sample container 153. The sample port 203 may include a connector valve 205 disposed in line with the sample flow path 214. The connector valve 205 may be configured to selectively (1) open to allow flow of liquid 123 through the sample port 203 and (2) close to prevent flow of liquid 123 through the sample port 203 as further described below. An anti-microbial coating 209 may be applied to one or more surfaces of the sampling system 350 to inhibit contamination of the liquid 123.

In some embodiments, the inlet port 201 and outlet port 202 may be arranged orthogonal to each other, and the inlet port 201 and the sample port 203 may be linearly arranged. In other embodiments, the inlet port 201 and outlet port 202 may be linearly arranged, and the sample port 203 may be arranged orthogonal to the inlet port 201 and outlet port 202. The connector 151 may be formed of a suitable plastic material such as polypropylene, polyethylene, polyvinyl chloride (PVC) or any other suitable material.

The connector 151 may include a connector septum 206 disposed within the sample port 203 so as to extend across the sample port 203. In some embodiments, the connector septum 206 may be located adjacent a proximal end 251A of the connector 151. The connector septum 206 forms a seal across the sample port 203, thereby preventing flow of liquid 123 through the sample port 203. In other words, the connector septum 206 is configured to define the “closed” state of the connector valve 205.

In some embodiments, the connector septum 206 may extend continuously across the sample port 203 to define the seal. In other embodiments, the connector septum 206 may include one or more deflectable flap portions (or pedal portions) extending at least partially across the sample port 203. The flap portions may be coupled to an inside surface of the connector 151. The flap portions may engage each other within the sample port 203 to define the seal across the sample port 203.

With reference to the FIGS. 2A and 2B, the sampling system 150 includes a coupling mechanism 216 configured to couple the connector 151 with the sample container 153. The coupling mechanism 216 includes corresponding coupling portions 216A, 216B included by the connector 151 and the sample container 153, respectively. In some embodiments, the coupling portions 216A, 216B may define an interference fit between the connector 151 and the sample container 153. In other embodiments, the coupling mechanism 216 may include corresponding threaded portions or corresponding latching components. The coupling mechanism 216 may also define a fluid seal between the connector 151 and the sample container 153. In some embodiments, the coupling portions 216A, 216B may include corresponding tapered portions to define a tapered interference fit, such as a Luer taper, for example.

The cap 152 is configured to the couple with the sample container 153 to provide a seal across an open end of the sample container 153. In some embodiments, the cap 152 may include a coupling portion 216C configured to correspond to the coupling portion 216B of the sample container 153. In use, the cap 152 may be applied to the sample container 153 to prevent liquid 123 from spilling from the sample container 153 during handling of the sample container 153.

With reference to FIG. 2B, in some embodiments, the connector body 207 may be configured to deform in response to an external force applied thereto, such as a pinching force 211, for example. In other embodiments, the external force may be a bending force. The deformation of the body 207 deflects, deforms, or displaces the septum 206 to defeat the seal across the sample port 203. In other words, the application of the pinching force 211 deflects the septum 206 to define the “open” configuration of the valve 205 so that the sample flow path 214 extends through the sample port 203 and into the sample container 153. In use, the clinician may initiate the drainage process and then apply the pinching force 211 to the connector 151 to open the valve 205 and obtain a sample of liquid 123 within the sample container 153.

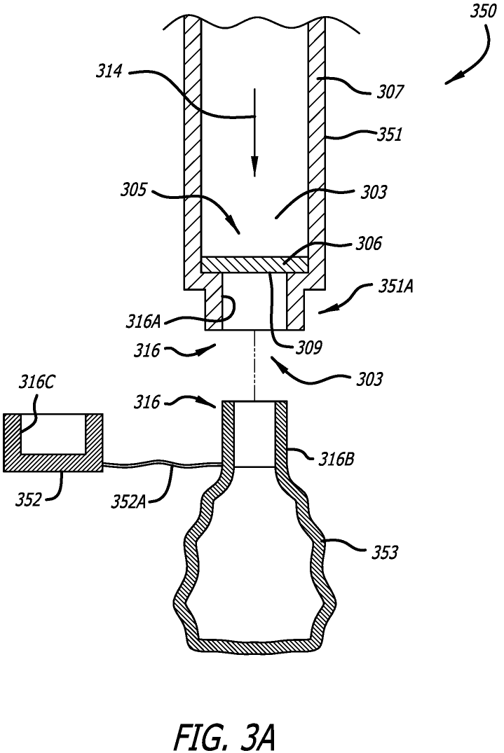

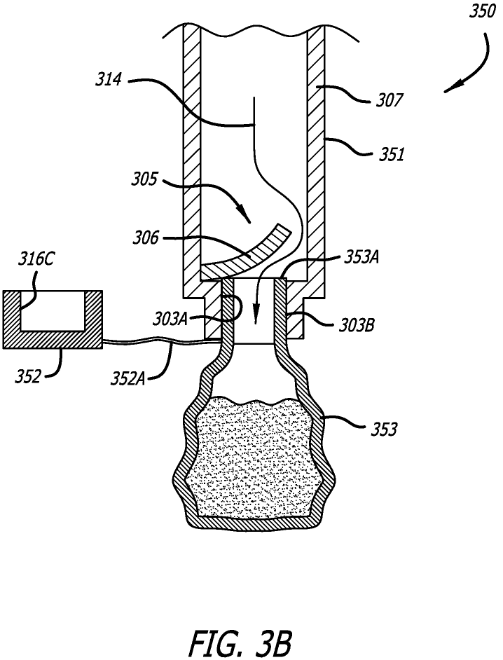

FIGS. 3A-3B illustrate a sampling system 350 in a disconnected state and a connected state, respectively. The sampling system 350 can, in certain respects, resemble components of the sampling system 150 described in connection with FIGS. 1-2B. It will be appreciated that all the illustrated embodiments may have analogous features. Accordingly, like features are designated with like reference numerals, with the leading digits incremented to “3.” For instance, the connector designated as “151” in FIGS. 1-2B, and an analogous connector is designated as “351” in FIGS. 3A-3B. Relevant disclosure set forth above regarding similarly identified features thus may not be repeated hereafter. Moreover, specific features of the sampling system 150 and related components shown in FIGS. 1-2B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the needle of FIGS. 3A-3B. Any suitable combination of the features, and variations of the same, described with respect to the sampling system 150 and components illustrated in FIGS. 1-2B can be employed with the sampling system 350 and components of FIGS. 3A-3B, and vice versa. This pattern of disclosure applies equally to further embodiments depicted in subsequent figures and described hereafter.

The connector 351 defines a sample flow path 314 along a sample port 303. In use, the liquid 123 may flow along the sample flow path 314, and through the sample port 303 to the sample container 353. The sample port 303 may include a connector valve 305 disposed in line with the sample port 303. The connector valve 305 may be configured to selectively (1) open to allow flow of liquid 123 through the sample port 303 and (2) close to prevent flow of liquid 123 through the sample port 303 as further described below.

The connector 351 may include a connector septum 306 disposed within the sample port 303 so as to extend across the sample port 303. In some embodiments, the connector septum 306 may be located adjacent a proximal end 351A of the connector 351. The connector septum 306 selectively forms a seal across the sample port 303, thereby preventing flow of liquid 123 through the sample port 303. In other words, the septum 306 is configured to define the “closed” state of the connector valve 305.

In some embodiments, the septum 306 may extended continuously across the sample port to define the seal. A portion of the septum 306 may be attached to an inside surface of the connector 351. The septum 306 may include an anti-microbial coating 309 to inhibit contamination of the liquid 123. In some embodiments, the anti-microbial coating 309 may be applied to other surfaces of the sampling system 350.

With reference to the FIGS. 3A and 3B, the sampling system 350 includes a coupling mechanism 316 configured to couple the connector 351 with the sample container 353. The coupling mechanism 316 includes corresponding coupling portions 316A, 316B included by the connector 351 and the sample container 353, respectively. In some embodiments, the coupling portions 316A, 316B may define an interference fit between the connector 351 and the sample container 353. In other embodiments, the coupling mechanism 316 may include corresponding threaded portions or corresponding latching components. The coupling mechanism 316 may also define a fluid seal between the connector 351 and the sample container 353. In some embodiments, the coupling portions 316A, 316B may include corresponding tapered portions to define a tapered interference fit, such as a Luer taper, for example.

The cap 352 is configured to the couple with the sample container 353 so as to provide a seal across an open end of the sample container 353. In some embodiments, the cap 352 may include a coupling portion 316C configured to correspond to the coupling portion 316B of the sample container 353. In some embodiments, the cap 352 may be attached to the sample container 353 via a tether 352A.

With reference to FIG. 3B, when the sampling container 353 is connected to the connector 351, a tip 353 of the sample container 353 may engage the septum 306 so as to deflect or deform the septum 306. The deflection of the septum 306 may defeat the seal across the sample port 303. In other words, connecting the sample container 353 to the connector 351 deflects the septum 306 to define the “open” configuration of the valve 305 so that the sample flow path 314 extends through the sample port 303 and into the sample container 353. In use, the clinician may initiate the drainage process and then connect the sample container to the connector 351 to open the valve 305 and obtain a sample of drainage liquid 123 within the sample container 353. Upon decoupling of the sample container 353 from the connector 351, the septum 306 may return to the non-deflected state to redefine the seal across the sample port 303, and thereby redefine the “closed” state of the valve 305.

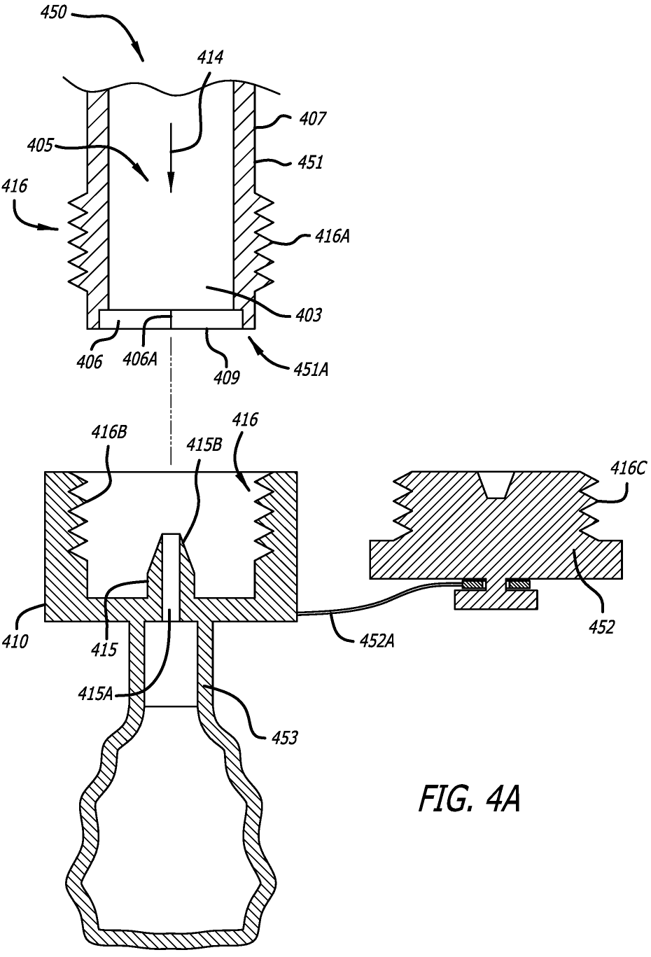

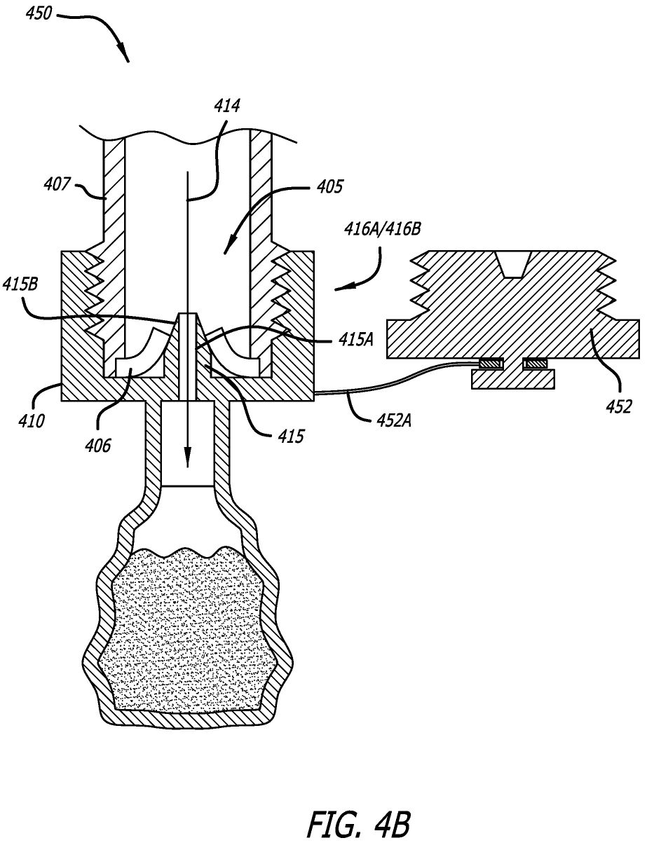

FIGS. 4A-4B illustrate a sampling system 450 in a disconnected state and a connected state, respectively. The sampling system 450 includes the connector 451, the cap 452, and the sample container 453. The connector 451 defines a sample flow path 414 through a sample port 403. The sample port 403 may include a connector valve 405 disposed in line with the sample flow path 414. The connector valve 405 may be configured to selectively (1) open to allow flow of liquid 123 through the sample port 403 and (2) close to prevent flow of liquid 123 through the sample port 403 as further described below.

The connector 451 may include a connector septum 406 disposed within the sample port 403 so as to extend across the sample port 403 at a proximal end 451A of the connector 451. The connector septum 406 selectively forms a seal across the sample port 403, thereby preventing flow of liquid 123 through the sample port 403. In other words, the septum 406 is configured to define the “closed” state of the connector valve 405. In some embodiments, the septum 406 may include a slit 406A extending through the septum 406.

In some embodiments, the septum 406 may extend continuously across the sample port to define the seal. A portion of the septum 406 may be attached to an inside surface of the connector 451. The septum 406 may include an anti-microbial coating 409 to inhibit contamination of the liquid 123. In some embodiments, the anti-microbial coating 409 may be applied to other surfaces of the sampling system 450.

With reference to the FIGS. 4A and 4B, the sampling system 450 includes a coupling mechanism 416 configured to couple the connector 451 with a frame 410 of the sample container 453. The coupling mechanism 416 includes corresponding threaded portions 416A, 416B included by the connector 451 and the container frame 410, respectively. As shown, the threaded portions 416A, 416B may define a threaded attachment between the connector 451 and the sample container 453.

The sampling container 453 includes a hollow spike 415 extending distally away from a bottom wall of the frame 410. The spike 415 defines a lumen 415A extending from a distal tip 415B to an interior of the sample container 453.

The cap 452 is configured to the couple with the sample container 453 to provide a seal across an open end of the sample container 453. In some embodiments, the cap 452 may include a coupling portion 416C configured to correspond to the coupling portion 416B of the sample container 453. In some embodiments, the cap 452 may be attached to the sample container 453 via a tether 452A.

With reference to FIG. 4B, when the sampling container 453 is connected to the connector 451, the tip 415B of the spike 415 may engage the septum 406 so as to deflect or deform the septum 406. The deflection of the septum 406 may defeat the seal across the sample port 403. In other words, connecting the sample container 453 to the connector 451 deflects the septum 406 to define the “open” configuration of the valve 405 so that the sample flow path 414 extends through the sample port 403 and into the sample container 453. In some embodiments, the spike 415 may pierce the septum 406 and in further embodiments, the spike 415 may extend through the slit 406A of the septum 406. In use, the liquid 123 (FIG. 1) may flow along the sample flow path 414, through the spike 415, and into the sample container 453. In use, the clinician may initiate the drainage process and then connect to the sample container 453 to the connector 451 to open the valve 405 and obtain a sample of drainage liquid 123 within the sample container 453. Upon decoupling of the sample container 453 from the connector 451, the septum 406 may return to the non-deflected state to redefine the seal across the sample port 403, and thereby redefine the “closed” state of the valve 405.

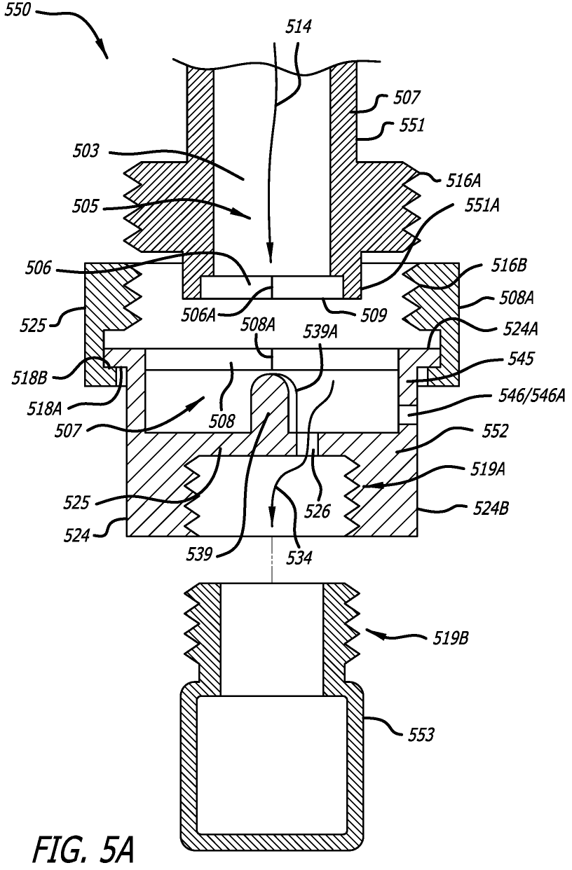

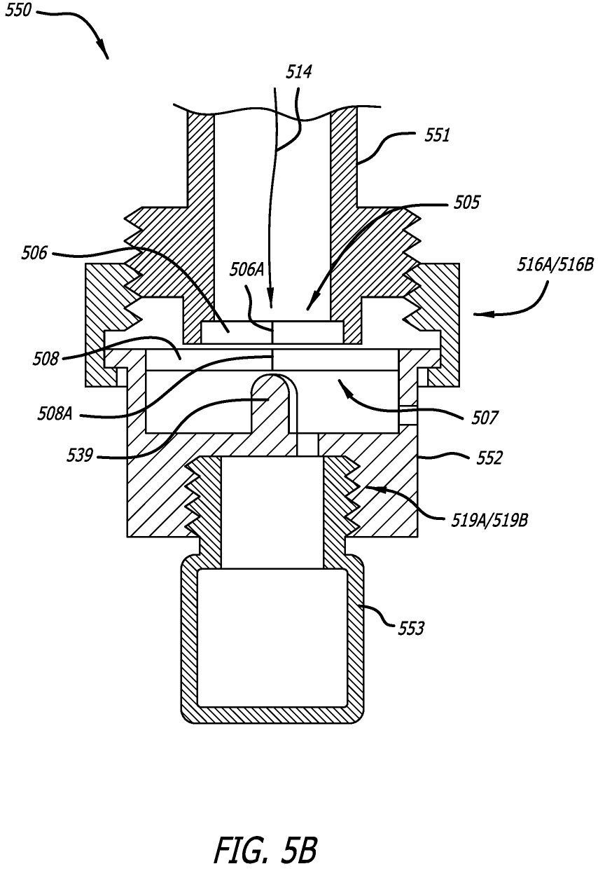

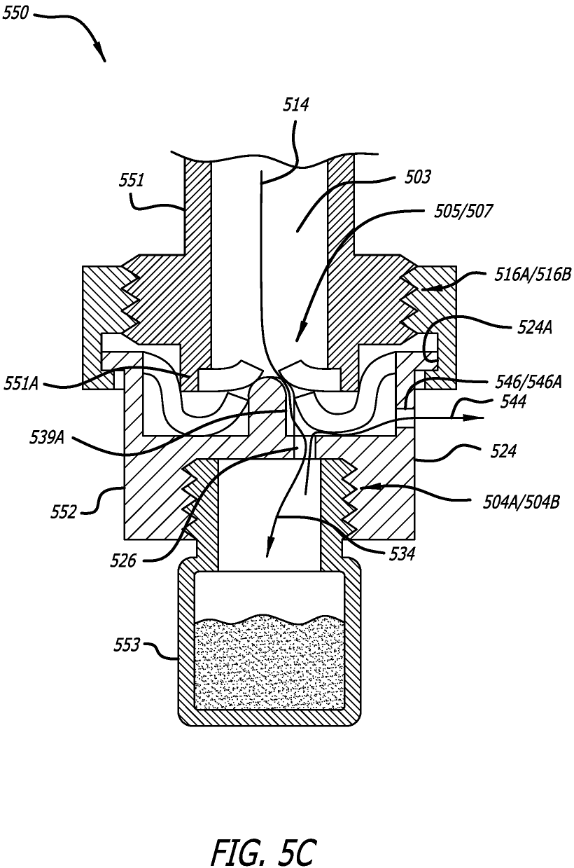

FIGS. 5A-5C illustrate the drainage liquid sampling system 550 including the connector 551, the cap 552, and the sample container 553. FIG. 5A shows the sampling system 550, i.e., the connector 551, the cap 552, and the sample container 553 in a disconnected state. FIG. 5B shows the connector 551 and the sample container 553 in a partially connected state. FIG. 5C shows the connector 551 and the sample container 553 in a fully connected state.

The connector 551 defines a sample flow path 514 extending through a sample port 503. The sample port 503 includes a connector valve 505 disposed in line with the sample flow path 514. The connector valve 505 may be configured to selectively (1) open to allow flow of liquid 123 through the sample port 503 and (2) close to prevent flow of liquid 123 through the sample port 503 as further described below.

The connector 551 includes a connector septum 506 disposed within the sample port 503 so as to extend across the sample port 503 at a proximal end 551A of the connector 551. The connector septum 506 selectively forms a seal across the sample port 503, thereby preventing flow of liquid 123 (FIG. 1) through the sample port 503. In other words, the septum 506 is configured to define the “closed” state of the connector valve 505. In some embodiments, the septum 506 includes a slit 506A extending through the septum 506.

The septum 506 extends continuously across the sample port to define the seal across the sample port 503. The septum 506 may be attached to an inside surface of the connector 551. The septum 506 may include an anti-microbial coating 509 to inhibit contamination of the liquid 123. In some embodiments, the anti-microbial coating 509 may be applied to other surfaces of the sampling system 550.

With further reference to FIG. 5A, the cap 552 is configured to couple with the sample container 553 via an included rotatable collar 525. The connector 551 and the collar 525 include corresponding threaded portions 516A, 516B to facilitate a threaded coupling between the connector 551 and the collar 525. The collar 525 is rotatably coupled to a body 524 of the cap 552 so that the collar 525 may rotate with respect to the body 524. The body 524 and the collar 525 include corresponding overlapping portions 518A, 518B, respectively, that constrain longitudinal displacement of the collar 525 with respect to the body 524.

The cap 552 includes a sample flow path 534 of the cap 552 extending from a distal end 524A to a proximal end 524B of the body 524. The flow path 534 includes one or more openings 526 extending through a center wall 525 of the body 524. A protrusion 539 extends distally away from the center wall 525. The body 524 and the sample container 553 include corresponding threaded portions 519A, 519B, respectively to facilitate threaded coupling of the cap 552 with the sample container 553.

The cap 552 may include a cap valve 507 disposed in line with the sample flow path 534. The cap valve 507 may be configured to selectively (1) open to allow flow of liquid 123 through the body 524 and (2) close to prevent flow of liquid 123 through the body 524 as further described below.

The cap 552 may include a connector septum 508 disposed across the flow path 534 at the distal end 524A of the body 524. The cap septum 508 selectively forms a seal across the flow path 534, thereby preventing flow of liquid 123 through the body 524. In other words, the cap septum 508 is configured to define the “closed” state of the cap valve 507. The cap septum 508 is configured to deflect or deform to defeat the seal across the sample flow path 534, thereby allowing flow of liquid 123 through the body 524. In other words, the cap septum 540 is also configured to define the “open” state of the cap valve 506 as described below. The septum 508 may include the anti-microbial coating 509 to inhibit contamination of the liquid 123.

In some embodiments, the cap septum 508 may include a slit 508A extending through the cap septum 508. The slit 540A is configured to transition between an “closed” configuration defining the “closed” state of the cap valve 506 as shown in FIGS. 5A and 5B, and an “open” configuration defining the “open” state of the cap valve 506 as shown in FIG. 5C.

In some embodiments, the cap 552 may include vent 546 extending through an external wall 545 of the body 524. The vent 546 may be configured to allow passage of air through the external wall 545 from the sample flow path 534 to the environment. The vent 546 may include a hydrophobic membrane 546A to prevent passage of liquid 123 through the vent 546. In use, the liquid 123 may flow into the sample container 553 displacing air in the sample container 553. The displaced air may flow out of the sample container 553 and through the vent 546.

FIG. 5B shows the sampling system 550 in a partially connected state. In the partially connected state, the corresponding threaded portions 519A, 519B are engaged so that the sample container is attached to the cap 552. The corresponding threaded portions 517A, 517B are partially engaged so that the cap 552 is attached to the connector 551. In the partially connected state, the connector septum 520 and the cap septum 540 may be disposed adjacent each other. In the partially connected state, one or both of the connector septum 506 and the cap septum 508 are in a non-deflected state so that either or both of the slits 506A, 508A are closed. In some embodiments, the connector septum 505 may be in contact with the cap septum 508 so as to form a fluid seal between the connector septum 506 and cap septum 508. In use, the clinician may selectively dispose the sampling system 550 in the partially connected state to facilitate flow of drainage liquid 523 from the catheter 110, through the connector 551 along the main flow path 513, and into the connection container 130 without obtaining a sample of the liquid 123.

FIG. 5C shows the sampling system 550 in a fully connected state. In the fully connected state, the corresponding threaded portions 519A, 519B are engaged so that the sample container is attached to the cap 552 and the corresponding threaded portions 517A, 517B are fully engaged. In the fully connected state, liquid 123 flows from the catheter 110, through the connector 551 along the main flow path 513 and into the connection container 130. Liquid 123 also flows through the connector 551 along the sample flow path 514, through the cap along the sample flow path 534, and into the sample container 553. Air from the sample container 553 may flow through the cap 552 along the air flow path 544 and through the vent 546 to the environment.

In the fully connected state, the connector 551 is proximally displaced relative to the cap 552, so that the proximal end 551A of the connector 551 is displaced proximal the distal end 524A of the body 524. As such, the cap septum 506 engages the protrusion 539 and the connector septum 506 engages the cap septum 508. The engagement with the protrusion 539 causes deflection of the both the connector septum 506 and the cap septum 508 sufficient to open the slits 506A, 508A. In other words, disposition of the sampling system 550 in the fully connected state opens the connector valve 505 and the cap valve 507 thereby allowing flow of liquid 123 into the sample container 553.

The septa 506, 508, and the protrusion 539 are correspondingly structured to prevent obstruction of the flow of liquid through the slits 506A, 508A by the protrusion 539. For example, a bottom surface of the cap septum 508 and/or a portion of the protrusion 539 may include protrusions, depressions, ridges, or troughs (e.g., the trough 539A) to avoid sealing contact of the cap septum 508 with the protrusion 539. In some embodiments, the protrusion 539 may be hollow so that the sample flow path 534 extends through the protrusion 539.

Methods of use may include the following steps of processes. A clinician may obtain a drainage system including a sampling system fixedly disposed in line with the drainage tube. In some embodiments, the clinician may obtain the sampling system independent from the drainage tube. The sampling system may or may not include the sample container connected to the cap. The sampling system may or may not include the cap connected to the connector. The cap may be connected to the connector. In some embodiments, the sampling system may be fully connected upon initiation of the drainage process. The clinician may transition the sampling system from the partially connected state to the fully connected state to allow liquid to flow into the sample container. The clinician may disconnect the cap from the connector. Upon disconnection, the connector valve and the cap valve may automatically close. The clinician may remove the cap from the sample container to access the sampled liquid. In some embodiments, the clinician may couple the sampling system to the drainage tube.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A drainage liquid sampling system, comprising:
a connector couplable with a drainage tube of a drainage system, the connector defining an inlet port, an outlet port, and a sample port, wherein:
the inlet port, the outlet port, and the sample port are in fluid communication with each other, and
drainage liquid flowing through the drainage tube flows through the connector from the inlet port to the outlet port; and
a sample container couplable with the sample port.

2. The system of claim 1, wherein the connector comprises a connector valve disposed in line with the sample port, the connector valve selectively configurable between an open state and a closed state, wherein drainage liquid is:
permitted to enter through the sample port when the connector valve is in the open state, and
prevented from exiting through the sample port when the connector valve is in the closed state.

3. The system of claim 2, wherein the connector valve is configured to:
automatically open upon coupling of the sample container with the connector, and
automatically close upon decoupling of the sample container from the connector.

4. The system of claim 2, wherein the connector valve comprises a connector septum disposed across the sample port.

5. The system of claim 4, wherein the connector septum comprises an anti-microbial coating.

6. The system of claim 1, further comprising a cap couplable with the sample container, the cap configured to sealably couple with an open end of the sample container.

7. The system of claim 6, wherein the cap comprises a cap valve selectively configurable between an open state and a closed state, wherein drainage liquid is:
permitted to enter into the sample container when the cap valve is in the open state, and
prevented from exiting the sample container when the cap valve is in the closed state.

8. The system of claim 6, wherein the cap is configured to couple with the connector.

9. The system of claim 8, wherein the cap valve is configured to automatically:
permit drainage liquid to enter the sample container upon coupling of the cap with the connector, and
prevent drainage liquid from exiting the sample container upon decoupling of the cap from the connector.

10. The system of claim 1, wherein at least a portion of the sampling system comprises an anti-microbial coating.

11. The system of claim 10, wherein the connector septum is deflectable, wherein the connector valve is in:
the open state when the connector septum is in a deflected state, and
the closed state when the connector septum is in a non-deflected state.

12. The system of claim 11, wherein the connector septum comprises a connector slit extending through the connector septum, the connector slit configurable between an open condition and a closed condition, wherein the connector slit is in:
the open condition when the connector septum is in the deflected state, and
the closed condition when the connector septum is in the non-deflected state.

13. The system of claim 6, wherein the cap valve comprises a cap septum disposed across an opening at a distal end of the cap.

14. The system of claim 13, wherein the cap septum is deflectable, wherein the cap valve is in:
the open state when the cap septum is in a deflected state, and
the closed state when the cap septum is in a non-deflected state.

15. The system of claim 14, wherein the cap septum comprises a cap slit extending through the cap septum, the cap slit configurable between an open condition and a closed condition, wherein the cap slit is in:
the open condition when the cap septum is in the deflected state, and
the closed condition when the cap septum is in the non-deflected state.

16. The system of claim 13, wherein:
the cap comprises a protrusion disposed within the opening of the cap, and
the protrusion is configured to deflect the cap septum and connector septum upon coupling of the cap with the connector.

17. The system of claim 13, wherein the cap septum comprises an anti-microbial coating.

18. A drainage system for draining a liquid from a patient, comprising:
a drainage tube configured to couple with a drainage catheter at a distal end of the drainage tube;
a collection container coupled to the drainage tube at a proximal end of the drainage tube; and
the sampling system of claim 1, the sampling system connected in line with the drainage tube.

19-25. (canceled)
